(19) United States
(12) Patent Application Publication
(10) Pub. No.: US 2006/0166928 A1
(43) Pub. Date: Jul. 27, 2006

(54) Hyaluronic acid derivative gel and method for preparing the same

(76) Inventors: Tae Seok Moon, Gyeonggi-do (KR); Jin Hoon Kim, Daejeon (KR); Jae Young Lee, Seoul (KR); Byung Hyuk Min, Incheon (KR); Kwang Yong Cho, Gyeonggi-do (KR)

Correspondence Address: CANTOR COLBURN, LLP, 55 GRIFFIN ROAD SOUTH, BLOOMFIELD, CT, 06002, US

(21) Appl. No.: 10/521,003

(22) PCT Filed: May 21, 2003
(86) PCT No.: PCT/KR03/00998

§ 371 Date: Nov. 21, 2005

(30) Foreign Application Priority Data
Jul. 26, 2002 (KR) 10-2002-0044261

(51) Int. Cl. A61K 31/728; C08B 37/00

(52) U.S. Cl. 514/54; 536/53

(57) ABSTRACT

The present invention relates to a hyaluronic acid derivative gel, obtained by amidation of a hyaluronic acid, or its cationic salt, and an amine group-containing saccharide compound, having excellent viscoelastic properties, and a method for preparing it. Especially, the hyaluronic acid derivative gel according to the present invention shows responses peculiar to heat and can be made to have various properties by heat treatment. The hyaluronic acid derivative gel according to the present invention can be used for a variety of purposes such as post-operative adhesion-preventing gel, material for wrinkle treatment, auxiliary material for plastic surgery, material for arthritis treatment, and drug delivery vehicle.

### FIELD OF THE INVENTION

The present invention relates to hyaluronic acid derivative gels, more particularly hyaluronic acid derivative gels which are formed by coupling an amine group-containing saccharide compound, having a variety of molecular weights, to a hyaluronic acid, having a variety of molecular weights, or a cationic salt thereof, via amidation reaction, and a method for preparing the same. The hyaluronic acid derivative gels according to the present invention have various different properties to heat, depending upon the amidation reaction condition and additional heat treatment.

### BACKGROUND OF THE INVENTION

Hyaluronic acid is a linear biocompatible polymer comprising linked repeating units of N-acetyl-D-glucosamine and D-glucuronic acid, which is present in high concentrations in the vitreous body of the eye, the synovial fluid of joints, rooster comb, etc. As used herein, the term “hyaluronic acid” sometimes refers to both hyaluronic acid and any of its cationic salts. The cationic salt of hyaluronic acid used in the present invention includes such inorganic salts as sodium hyaluronate and potassium hyaluronate and such organic salts as tetrabutylammonium hyaluronate, but is not limited thereto.

Hyaluronic acid derivatives have been widely developed to be used as post-operative adhesion-preventing films or gels, menials for wrinkle treatment, materials for plastic surgery, materials for arthritis treatment, vehicles for drug delivery system, etc. Especially, increasing attention has been focused on hyaluronic acid derivative gel, due to peculiar properties thereof in many application fields. For example, U.S. Pat. No. 5,356,883 discloses hyaluronic acid derivative gel in which carboxyl group of hyaluronic acid, or a salt thereof, has been modified to O-acyl or N-acyl ureas by using various kinds of carbodiimides. U.S. Pat. No. 5,827,937 discloses a cross-linked polysaccharide gel obtained by cross-linking reaction consisting of two steps. Further, U.S. Pat. No. 5,399,351 discloses methods for preparing gels having various properties.

### SUMMARY OF THE INVENTION

One object of the present invention is to provide hyaluronic acid derivative gels in which an amine group containing saccharide compound is attached to a hyaluronic acid by amidation.

Another object of the present invention is to provide hyaluronic acid derivative gels having various different properties to heat, depending upon reaction conditions.

A further object of the present invention is to provide a method for preparing hyaluronic acid derivative gels having various different properties by heat treatment.

Hyaluronic acid derivative gels in accordance with the present invention are prepared by bonding a hyaluronic acid, having a variety of molecular weights, and amine group-containing saccharide compounds, having a variety of molecular weights, via amidation. These hyaluronic acid derivative gels have excellent viscoelastic properties and can thus be applied to many uses. Especially, the hyaluronic acid derivative gels of the present invention are materials showing heat-specific responses and can be made to gels having various different properties by heat treatment. Moreover, the present invention provides various hyaluronic acid derivatives having various properties to heat, which can be prepared depending upon the amidation reaction conditions.

Additionally, since the hyaluronic acid derivative gels according to the present invention have covalent bonds, i.e., amide bonds, between hyaluronic acid and an amine group-containing saccharide compound, they can stand several conditions in vivo. These gels are novel biocompatible materials having largely different properties from the existing hyaluronic acid derivatives synthesized using carbodiimide compound.

A method for preparing hyaluronic acid derivative gels in accordance with the present invention comprises mixing a solution of hyaluronic acid and a solution of amine group-containing saccharide compound to form ionic bonds between them, then reacting the anionic carboxyl groups of hyaluronic acid with the cationic amine groups of saccharide compound by using an agent for activating carboxyl group, and washing the reactant with water or an acid solution to yield the refined material, followed by separating it and then drying. In other words, the hyaluronic acid derivative gels can be prepared through the procedure comprising a step of mixing/agitating hyaluronic acid and an amine group-containing saccharide compound, a step of activating the carboxyl group of the hyaluronic acid, and a step of reacting the activated carboxyl group of the hyaluronic acid with the amine group of the saccharide compound. The above procedure has advantages that the reaction process is easy, the separation step is simple, and no harmful organic solvents are used.

The hyaluronic acid, or its cationic salt, used in the present invention is preferably one or more selected from a group consisting of sodium hyaluronate, potassium hyaluronate, ammonium hyaluronate, calcium hyaluronate, magnesium hyaluronate and tetrabutylammonium hyaluronate.

A final reaction concentration of said hyaluronic acid is preferably in the range of between 0.05 mg/ml and 50 mg/ml. A “final reaction concentration,” as that term is used herein, of a certain component (A) means a concentration of the component (A) in a total reaction solution also containing other components (B, C . . . ) in addition to the component (A).

An average molecular weight of said hyaluronic acid is preferably in the range of between 500,000 and 5,000,000.

Said amine group-containing saccharide compound is one or more selected from a group consisting of chitosan, chitosan derivatives, deacetylated hyaluronic acid and deacetylated hyaluronic acid derivatives.

Said amine group-containing saccharide compound is preferably added in an amount such that the ratio of the amine group to the carboxyl group of hyaluronic acid is in the range of between 0.01 and 100 (molar equivalents of the amine group to 1 molar equivalent of the carboxyl group).

As mentioned earlier, activation of the carboxyl group can be induced using an activating agent. The activating agent is not specifically limited as long as it can activate the carboxyl group of hyaluronic acid and is soluble in water, but preferably is a mixture of one or more compounds, as a main agent, selected from a group consisting of 1-alkyl-3-(3-dimethylaminopropyl)carbodiimides (alkyl herein is alkyl of 1-10 carbon atoms), 1-ethyl-3-(3-(trimethylammonio)propyl)carbodiimide (“ETC”) and 1-cyclohexyl-3-(2-morpholinoethyl)carbodiimide (“CMC”), and one or more compounds, as an auxiliary agent, selected from a group consisting of 1-hydroxybenzotriazole (“HOBt”), 3,4-dihydro-3-hydroxy-4-oxo-1,2,3-benzotriazine (“HOOBt”), 1-hydroxy-7-azabenzotriazole (“HOAt”), N-hydroxysuccinimide (“NHS”) and sulfo-NHS. The activation agent is more preferably a mixture of 1-ethyl-3-(3-dimethylaminopropyl)carbodiimide hydrochloride (“EDC”) and NHS.

The main activating agent is preferably added in a final reaction concentration of 0.01 mg/ml to 20 mg/ml. The auxiliary activating agent is also preferably added in a final reaction concentration of 0.1 mg/ml to 20 mg/ml.

Hyaluronic acid derivative gels of the present invention are materials showing heat-specific responses and can thus be made to have a variety of properties by heat treatment. The temperature for said heat treatment is preferably in the range of between 25° C. and 130° C., more preferably 40° C. to 80° C. The duration of said heat treatment is preferably in the range of between 0.5 hour and 144 hours. Heat treatment can be performed by various ways, for example, gradually heating a gel, heating a gel to a certain temperature and then maintaining at that temperature for a specific time, heating a gel to instantaneously change its temperature, etc.

The product obtained from the amidation reaction in accordance with the present invention can be separated and/or refined by well-known methods in the art to which the present invention pertains. These separation and refinement methods include distillation (under atmospheric pressure or reduced pressure), recrystallization, column chromatography, ion-exchange chromatography, gel chromatography, affinity chromatography, thin-layer chromatography, phase separation, solvent extraction, dialysis, washing, etc. Each refinement may be performed after each reaction or after series of reactions.

Hereinafter, the present invention will be described in detail by EXAMPLES, but the scope of the present invention is not limited thereto.

### DETAILED DESCRIPTION OF PREFERRED EMBODIMENTS

### EXAMPLE 1

### Preparation of Hyaluronic Acid Derivative Gel with Chitosan Coupled Thereto

To produce a hyaluronic acid derivative gel to which chitosan is coupled, 1 ml of a stock solution containing 40 mg of chitosan (average molecular weight: 300 to 1,600; EugenBio) was added to 34 ml of a stock solution containing 200 mg of sodium hyaluronate (average molecular weight: 500,000 to 2,500,000; LGCI), to form a final solution having a final reaction concentration of chitosan of 1.0 mg/ml and a final reaction concentration of sodium hyaluronate of 5.0 mg/ml, and then stirred. To this mixture, added were 2.5 ml of a stock solution containing 125 mg of EDC and 2.5 ml of a stock solution containing 150 mg of NHS to final reaction concentrations of 3.125 mg/ml and 3.750 mg/ml, respectively, and then stirred. After addition of EDC and NHS, reaction was carried out at 25° C. for 3 hours, thereby obtaining a gel of high viscoelasticity. For comparison with the above, another solution was prepared in the same manner as the above process except that no chitosan was added, thereby not forming any gel.

### EXAMPLES 2 TO 5

### Preparation of Hyaluronic Acid Derivative Gel with Chitosan Coupled Thereto and Measurement of Swelling Ratio

For convenience of explanation, hereinafter, the amount of components is represented as only a final reaction concentration.

To provide a hyaluronic acid derivative gel to which chitosan is coupled, a solution containing chitosan (average molecular weight: 300 to 1,600; EugenBio) in several final reaction concentrations as shown in Table 1 was added to a solution containing sodium hyaluronate (average molecular weight: 2,500,000 to 5,000,000; LGCI) in a final reaction concentration of 5.0 mg/ml, and the mixture was then stirred. To the inure, added were EDC in a final reaction concentration of 0.625 mg/ml and NHS in a final reaction concentration of 0.750 mg/ml and then stirred. After addition of EDC and NHS, reaction was carried out at 25° C. for 17 hours. The concentration of sodium chloride was then adjusted to 1 M. Ethanol equal to the volume of the reaction solution was added to precipitate hyaluronic acid derivative. The precipitate was separated from the reaction solution, washed and dried. Water was added to the dried hyaluronic acid derivative, with the latter being adjusted to a concentration of 10 mg/ml, thereby obtaining a suspension solution consisting of gel. Only gel-phase product was separated from the suspension solution, then some water on the surface of gel was removed to measure the weight of gel (Wwet). After measurement of weight, the gel was heated at 120° C. for 45 minutes for drying to measure the weight of the dried hyaluronic acid derivative (Wdry). The swelling ratio of the hyaluronic acid derivative gel was calculated based upon the following formula, and the result is given in Table 1.
Swelling Ratio=Wwet/Wdry

TABLE 1
Swelling ratio of hyaluronic acid
derivative gel of EXAMPLES 2 to 5

| | Sodium hyaluronate | Chitosan | Swelling ratio |
|---|---|---|---|
| Ex. | (mg/ml) | (mg/ml) | (Wwet/Wdry) |
| 2 | 5.0 | 0.125 | 18.8 |
| 3 | 5.0 | 0.250 | 30.8 |
| 4 | 5.0 | 0.500 | 58.1 |
| 5 | 5.0 | 1.000 | >100 |

### EXAMPLES 6 TO 9

### Preparation of Hyaluronic Acid Derivative Gel with Chitosan Coupled Thereto and Measurement of Complex Viscosity

To produce hyaluronic acid derivative gel to which chitosan is coupled, a solution containing chitosan (average molecular weight: 300 to 1,600; EugenBio) in a final reaction concentration of 1.0 mg/ml was added to a solution containing sodium hyaluronate (average molecular weight: 500,000 to 2,500,000; LGCI) in a final reaction concentration of 5.0 mg/ml, and the mixture was then stirred. To the mixture, EDC and NHS were added in several final reaction concentrations as shown in TABLE 1, respectively. After addition of EDC and NHS, reaction was carried out at 25° C. for 17 hours. The concentration of sodium chloride was then adjusted to 1 M. Ethanol equal to the volume of the reaction solution was added to precipitate a hyaluronic acid derivative to which chitosan was coupled. The precipitate was separated from the reaction solution, washed and then dried. Water was applied to the precipitate to adjust the concentration of hyaluronic acid derivative to 10 mg/ml. As a result, the products were obtained having various phases as shown in TABLE 2.

Complex viscosities of the reaction mixtures in the end of the reaction were measured at 0.1 Hz and 25° C. with a rheometer (PAAR PHYSICA) and values obtained thus are described in TABLE 2.

TABLE 2
Complex viscosity and material phase of hyaluronic acid
derivative of EXAMPLES 6 to 9 (0.1 Hz, 25° C.)

| | | | Complex | Material phase after |
|---|---|---|---|---|
| | EDC | NHS | viscosity | addition of water |
| Ex. | (mg/ml) | (mg/ml) | (cP) | (10 mg/ml) |
| 6 | 0.000 | 0.000 | 520 | Solution |
| 7 | 0.125 | 0.150 | 560 | Suspension consisting of |
| | | | | minute gels |
| 8 | 0.625 | 0.750 | 1,200 | Suspension consisting of |
| | | | | small gels |
| 9 | 3.125 | 3.750 | 5,000 | One lump of gel |

### EXAMPLE 10

### Preparation of Deacetylated Hyaluronic Acid Derivative Gel

When hyaluronic acid is heated at low or high pH, deacetylation occurs to form amine groups having a high reactivity. For deacetylation, hyaluronic acid was reacted with 0.2 N to 10 N NaOH at 25° C. to 50° C. for 1 hour to 30 hours. As a result, deacetylated hyaluronic acids were obtained with degrees of deacetylation of 1% to 40%. To a solution of the deacetylated hyaluronic acid in a final reaction concentration of 10 mg/ml, added were a solution of EDC in a final reaction concentration of 2.4 mg/ml and a solution of NHS in a final reaction concentration of 2.9 mg/ml, then reacted at 25° C. for 3 hours. After refinement of the product, a gel was obtained.

### EXAMPLE 11

### Preparation of Hyaluronic Acid Derivative Gel with Deacelyated Hyaluronic Acid Coupled Thereto

A solution of deacetylated hyaluronic acid with a degree of deacetylation of 1% to 40% was mixed with a solution of hyaluronic acid (average molecular weight: 2,500,000 to 5,000,000) in a final reaction concentration of 0.5 mg/ml, respectively, to make a mixed solution. EDC in a final reaction concentration of 0.2 mg/ml and NHS in a final reaction concentration of 0.24 mg/ml were added to the mixed solution and reaction was then carried out at 25° C. for 3 hours. After termination of the reaction, the reactant was refined and dried to obtain the hyaluronic acid derivative gel with deacetylated hyaluronic acid coupled thereto.

### EXPERIMENT 1

### Measurement of Thermal Characteristics of Hyaluronic Acid Derivative Gel With Chitosan Coupled Thereto—1

To determine the thermal characteristic of the hyaluronic acid derivative gels to which chitosan is coupled, obtained in EXAMPLES 5, 7 and 8, the rheology of each gel was measured, with increasing the temperature in the range of 25° C. to 75° C., at 0.1 Hz, with a rheometer. The results are described in TABLES 3 to 5.

The hyaluronic acid derivative gel obtained in EXAMPLE 5 showed a rapid increase in viscoelasticity starting from about 60° C., and generally a very high elasticity. The hyaluronic acid derivative gel obtained in EXAMPLE 7 showed a decrease in viscoelasticity as the temperature increased, and also showed a higher viscosity than elasticity. Meanwhile, the hyaluronic acid derivative gel obtained in EXAMPLE 8 showed almost no variation in its viscoelasticity in the range of 25° C. to 75° C., thereby confining that no change in the physical structure thereof occurs depending upon the change of temperature.

TABLE 3
Rheology of hyaluronic acid derivative gel of
EXAMPLE 5 depending upon temperature (0.1 Hz)

| Temperature | Complex | Storage | Loss |
|---|---|---|---|
| (° C.) | viscosity (cP) | modules (Pa) | modules (Pa) |
| 25 | 51,000 | 30 | 11 |
| 30 | 49,000 | 29 | 11 |
| 35 | 46,000 | 27 | 10 |
| 40 | 42,000 | 25 | 9 |
| 45 | 37,000 | 22 | 8 |
| 50 | 37,000 | 22 | 7 |
| 55 | 53,000 | 33 | 6 |
| 60 | 56,000 | 35 | 5 |
| 65 | 496,000 | 310 | 38 |
| 70 | 1,130,000 | 706 | 83 |
| 75 | 13,741,000 | 8,226 | 2,665 |

TABLE 4
Rheology of hyaluronic acid derivative gel of
EXAMPLE 7 depending upon temperature (0.1 Hz)

| Temperature | Complex | Storage | Loss |
|---|---|---|---|
| (° C.) | viscosity (cP) | modules (Pa) | modules (Pa) |
| 25 | 980 | 0.1270 | 0.603 |
| 30 | 833 | 0.0996 | 0.515 |
| 35 | 713 | 0.0782 | 0.442 |
| 40 | 552 | 0.0616 | 0.342 |
| 45 | 467 | 0.0467 | 0.290 |
| 50 | 416 | 0.0393 | 0.259 |
| 55 | 348 | 0.0339 | 0.216 |
| 60 | 312 | 0.0385 | 0.193 |
| 65 | 277 | 0.0319 | 0.171 |
| 70 | 249 | 0.0386 | 0.152 |
| 75 | 244 | 0.0545 | 0.144 |

TABLE 5
Rheology of hyaluronic acid derivative gel of
EXAMPEL 8 depending upon temperature (0.1 Hz)

| Temperature | Complex | Storage | Loss |
|---|---|---|---|
| (° C.) | viscosity (cP) | modules (Pa) | modules (Pa) |
| 25 | 16,000 | 9.8 | 2.55 |
| 30 | 16,600 | 10.1 | 2.46 |
| 35 | 16,800 | 10.3 | 2.34 |
| 40 | 16,900 | 10.4 | 2.28 |
| 45 | 17,200 | 10.6 | 2.22 |
| 50 | 17,500 | 10.8 | 2.18 |
| 55 | 17,500 | 10.8 | 2.18 |
| 60 | 17,600 | 10.9 | 2.04 |
| 65 | 17,800 | 11.0 | 1.99 |
| 70 | 17,700 | 11.0 | 1.98 |
| 75 | 17,400 | 10.8 | 1.92 |

### EXPERIMENT 2

### Measurement of Thermal Characteristics of Hyaluronic Acid Derivative Gel with Chitosan Coupled Thereto—2

Hyaluronic acid derivative gel suspensions obtained in EXAMPLES 2, 3 and 4 were maintained at 60° C. for 36 hours, which resulted in gels of a high viscoelasticity. The complex viscosity of each gel was measured at 25° C. and 0.02 Hz using a rheometer and the result is described in TABLE 6.

TABLE 6
Complex viscosity of hyaluronic acid derivative
gel with chitosan coupled thereto (0.02 Hz)

| Ex. | Complex viscosity (cP) |
|---|---|
| 2 | 475,000 |
| 3 | 710,700 |
| 4 | 127,610 |

### EXPERIMENT 3

### Formation of Hyaluronic Acid Derivative Gel by Various Heat Treatments

Hyaluronic acid derivatives produced in EXAMPELS 1 to 5 and 7 to 9 were heat-treated at 25° C. to 130° C. for 0.1 hour to 72 hours, which resulted in gels, gel suspensions or solutions, having the rheology as follows:

Complex viscosity at 0.01 Hz to 0.1 Hz=100 cP to 20,000,000 cP

Storage modules at 0.01 Hz to 0.1H=0 Pa to 20,000 Pa

Loss modules at 0.01 Hz to 0.1 Hz=0 Pa to 5000 Pa

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

### INDUSTRIAL APPLICABILITY

As described above, the hyaluronic acid derivative gel according to the present invention, resulting from the reaction of hyaluronic acid and a saccharide compound containing amine groups, is a biocompatible material able to withstand various in vivo conditions due to covalent bonds thereof. Moreover, the hyaluronic acid derivative gel can be made through an easy reaction and simple separation process, using no harmful organic solvents, has a very good viscoelastic properties and can thus be used for various purposes such as post-operative adhesion-preventing gel, material for wrinkle treatment material for plastic surgery, material for arthritis treatment, and drug delivery vehicle. Especially, by using various reaction conditions, the hyaluronic acid derivatives can be made having various different properties to heat. Furthermore, these hyaluronic acid derivatives can be made in the form of gels, showing various and peculiar characteristics to heat, by various heat treatments.

1. A method for preparing a hyaluronic acid derivative gel, comprising the following steps:
(a) mixing a hyaluronic acid, or its cationic salt, and a saccharide compound containing amine groups, and then agitating;
(b) activating the carboxyl group of the hyaluronic acid or its cationic salt; and
(c) reacting the activated carboxyl group of the hyaluronic acid, or its cationic salt, with the amine group of the saccharide compound.

2. The method according to claim 1, wherein the cationic salt of hyaluronic acid is one or more selected from a group consisting of sodium hyaluronate, potassium hyaluronate, ammonium hyaluronate, calcium hyaluronate, magnesium hyaluronate, and tetrabutylammonium hyaluronate.

3. The method according to claim 1, wherein the final reaction concentration of hyaluronic acid, or its cationic salt, is in the range of between 0.05 mg/ml and 50 mg/ml.

4. The method according to claim 1, wherein the average molecular weight of hyaluronic acid, or its cationic salt, is in the range of between 500,000 and 5,000,000.

5. The method according to claim 1, wherein the amine group-containing saccharide compound is one or more selected from a group comprising of chitosan, chitosan derivatives, deacetylated hyaluronic acid, and deacetylated hyaluronic acid derivatives.

6. The method according to claim 1, wherein said saccharide compound containing amine groups is added in such an amount that the ratio of the amino group to the carboxyl group of the hyaluronic acid is in the range of 0.01:1 to 100:1.

7. The method according to claim 1, wherein activation of the carboxyl groups is accomplished by adding one or more agents for activating carboxyl groups.

8. The method according to claim 7, wherein activation of the carboxyl groups is accomplished by adding one or more compounds, as a main agent, selected from a group consisting of 1-alkyl-3-(3-dimethylaminopropyl)carbodiimides (alkyl herein is alkyl of 1-10 carbon atoms), 1-ethyl-3-(3-(trimethylammonio)propyl)carbodiimide (“ETC”) and 1-cyclohexyl-3-(2-morpholinoethyl)carbodiimide (“CMC”), and one or more compounds, as an auxiliary agent, selected from a group consisting of 1-hydroxybenzotriazole (“HOBt”), 3,4-dihydro-3-hydroxy-4-oxo-1,2,3-benzotriazine (“HOOBt”), 1-hydroxy-7-azabenzotriazole (“HOAt”), N-hydroxysuccinimide (NHS), and sulfo-NHS.

9. The method according to claim 8, wherein the main activation agent is 1-ethyl-3-(3-dimethylaminopropyl)carbodiimide hydrochloride (“EDC”) and the auxiliary activation agent is NHS.

10. The method according to claim 9, wherein EDC is added in a final reaction concentration of between 0.01 mg/ml and 20 mg/ml.

11. The method according to claim 9, wherein NHS is added in a final reaction concentration of between 0.1 mg/ml and 20 mg/ml.

12. The process according to claim 1, further including a step of heat-treating the hyaluronic acid derivative gel produced in step (c) at 25° C. to 130° C. for 0.5 hour 144 hours.

13. A hyaluronic acid derivative gel produced by the method in claim 1.
